(19) United States
(12) Patent Application Publication
(10) Pub. No.: US 2024/0261199 A1
(43) Pub. Date: Aug. 8, 2024

(54) SHAVING AID COMPOSITION INCLUDING A NATURAL WEAR RESISTANCE AGENT

(71) Applicant: EDGEWELL PERSONAL CARE BRANDS, LLC, CHESTERFIELD, MO (US)

(72) Inventors: Matthias Niggemann, Wuppertal (DE); Linda Low, Naugatuck, CT (US)

(21) Appl. No.: 18/565,288

(22) PCT Filed: Jun. 14, 2022
(86) PCT No.: PCT/US2022/033387

§ 371 Date: Nov. 29, 2023

Related U.S. Application Data
(60) Provisional application No. 63/210,233, filed on Jun. 14, 2021.

(51) Int. Cl. A61K 8/36; A61K 8/73; A61Q 9/02

(52) U.S. Cl. CPC A61K 8/361; A61K 8/73; A61Q 9/02; A61K 2800/30

(57) ABSTRACT

A shaving aid composition includes a soap base, such as an extruded soap base or a poured soap base, and a natural wear resistant agent selected from one or more polysaccharides. The one or more polysaccharides may include, for example, an algae extract, a seed extract, a plant exudate, a microorganism exudate, a fruit extract, a gelling agent of animal origin, or combinations thereof. In one example, the one or more polysaccharides include agar, xanthan gum, or combinations thereof. Additionally, the shaving aid composition may be substantially anhydrous and solid. Moreover, the shaving aid composition may be incorporated into a razor. For example, the shaving aid composition may be incorporated into a razor as a shaving aid body, a shaving aid strip, and the like, located on, in, or adjacent to a razor cartridge of the razor.

### CROSS-REFERENCE TO RELATED APPLICATIONS

This application claims priority to International Application No. PCT/US2022/033387, filed on Jun. 14, 2022, which claims priority to U.S. Provisional Patent Application No. 63/210,233, filed on Jun. 14, 2021, the contents of which are incorporated herein by reference in their entireties.

### TECHNICAL FIELD

Embodiments relate to a shaving aid composition including a soap base and a natural wear resistance agent to provide enhanced wear resistance of the shaving aid composition.

### BACKGROUND

Technologies are available to enhance shave quality. For example, a shave gel may use gelling agents in relatively large amounts (e.g., >20 wt. %) and may co-exist with relatively large amounts of water to form gels. A shave gel, however, may improve lubricity at the expense of convenience. Alternatively, a shaving aid composition may be located on a razor to improve lubricity, glide, etc. The shaving aid composition may have relatively low water content and relatively high soap content. However, such shaving aid compositions wear over time from repeated use. Synthetic lubricant polymers, such as polyethylene oxide (PEO), have been proposed for wear but can operate at the expense of shave performance (e.g., stringiness). Meanwhile, silicone polymers may cause sustainability concerns. Moreover, semi-solid esters may not be as effective themselves absent PEO or silicone polymers. Thus, there is considerable room for improvement to provide a shaving aid composition with a sustainable and/or natural wear resistance agent having good shave performance.

### SUMMARY

Embodiments relate to a shaving aid composition including a soap base and a natural wear resistance agent selected from a polysaccharide. In embodiments, the polysaccharide may include one or more polymeric carbohydrates composed of monosaccharide units bound together by glycosidic linkages.

The soap base may include a C₁₂-C₂₂ fatty acid, salt thereof, or combinations thereof. For example, the soap base may include lauric acid, myristic acid, palmitic acid, stearic acid, linolenic acid, salts thereof, or combinations thereof. Salts may include, for example, sodium salts, potassium salts, lower alkanolamine salts, or combinations thereof.

In any of the preceding embodiments, the soap base may include an additive such as, without limitation, a skin care ingredient, a surfactant, a skin conditioning agent, an absorbent, a coloring agent, a fragrance, or combinations thereof.

In any of the preceding embodiments, the soap base may be selected from an extruded soap base or a poured soap base. The soap base may, however, be manufactured by any traditional process known in the art without departing from the broader aspects of the present invention.

In any of the preceding embodiments, the soap base may be present in an amount from about 50.0 wt. % to about 99.5 wt. %, based on the total weight of the shaving aid composition. For example, the soap base may be present in an amount from about 75.0 wt. % to about 99.5 wt. %, based on the total weight of the shaving aid composition.

In any of the preceding embodiments, the polysaccharide may be selected from an algae extract, a seed extract, a plant exudate, a microorganism exudate, a fruit extract, a gelling agent of animal origin, or combinations thereof. In one example, the polysaccharide may include agar and the soap base may be selected from an extruded soap base or a poured soap base. Additionally, the polysaccharide may intentionally exclude guar gum, gummi arabicum, locust bean gum, and/or xanthan gum. In another example, the polysaccharide may include xanthan gum and the soap base may be selected from a poured soap base. Moreover, the shaving aid composition may exclude a synthetic lubricant polymer.

In any of the preceding embodiments, an individual polysaccharide or a combination of polysaccharides may be present in an amount from about 0.25 wt. % to about 15.0 wt. %, based on the total weight of the shaving aid composition. For example, the individual polysaccharide or the combination of polysaccharides may present in an amount from about 0.5 wt. % to about 3.0 wt. %, based on the total weight of the shaving aid composition.

In any of the preceding embodiments, the shaving aid composition may be substantially anhydrous, hydrous, and/or solid. The shaving aid composition may, for example, include water in an amount less than 20.0 wt. %, based on a total weight of the shaving aid composition, as a substantially anhydrous shaving aid composition. For example, the shaving aid composition may include water in an amount of about 15.0 wt. % or less. The shaving aid composition may, however, include water in an amount greater than 20.0 wt. %, based on a total weight of the shaving aid composition, as a substantially hydrous shaving aid composition. Additionally or alternatively, the shaving aid composition may be a collection of atoms or molecules held together so, under constant dry conditions, they maintain a defined shape and/or size as a substantially solid shaving aid composition.

Embodiments further relate to a razor including the shaving aid composition in any of the preceding embodiments. The shaving aid composition may, for example, be incorporated into the razor as a shaving aid body mounted on or adjacent to a razor cartridge of the razor. The shaving aid composition may also be incorporated into the razor as a shaving aid strip mounted in or on a razor cartridge of the razor. The shaving aid composition may, however, be incorporated into a razor in any other form or configuration known in the art without departing from the broader aspects of the present invention.

Embodiments further relate to a method to make the shaving aid composition in any of the preceding embodiments. For example, the method may include selecting the soap base, selecting the natural wear resistance agent, and combining the soap base and the wear resistance agent to form the shaving aid composition. Thus, for example, the method may include selecting an extruded soap base or a poured soap base. Moreover, the method may include selecting the polysaccharide based on the soap base selected. Additionally or alternatively, the method may include selecting the polysaccharide based on the stability at specific conditions, such as at the final pH-value of the shaving aid composition.

Embodiments further relate to a method to use the shaving aid composition in any of the preceding embodiments. For example, the method to use the shaving aid composition may include contacting the shaving aid composition with a solvent (e.g., water) to activate the shaving aid composition (e.g., to cause lubrication, etc.). The method may also include contacting the shaving aid composition with a keratinous surface or a hirsute surface to cause abrasion which can activate or aid on the activation of the shaving aid composition.

### BRIEF DESCRIPTION OF THE DRAWINGS

The features of the disclosure believed to be novel and the elements characteristic of the invention are set forth with particularity in the appended claims. The figures are for illustration purposes only and are not drawn to scale. The disclosure itself, however, both as to organization and method of operation, can best be understood by reference to the description of the preferred embodiment(s) which follows, taken in conjunction with the accompanying drawings in which:

FIG. 1 is a side elevation view of a shaving aid composition incorporated into a razor as a shaving aid body on or adjacent to a razor cartridge according to embodiments;

FIG. 2 is a perspective view of a replacement cartridge including a shaving aid body formed from a shaving aid composition according to embodiments; and

FIG. 3 is a perspective view of a replacement cartridge including a shaving aid body and a shaving aid strip formed from a shaving aid composition according to embodiments.

### DETAILED DESCRIPTION

Embodiments can comprise, consist of, and consist essentially of the features and/or steps described herein, as well as any of the additional or optional ingredients, components, steps, or features described herein or would otherwise be appreciated by one of skill in the art. It is to be understood that all concentrations disclosed herein are by weight percent (wt. %.) based on a total weight of the composition unless otherwise indicated. Where appropriate, the International Nomenclature of Cosmetic Ingredients (INCI) name of ingredients/components is provided. In this regard, the term “(and)” is an INCI designation for a mixture of listed components separated by the term. Any numerical range recited herein is intended to include all sub-ranges subsumed therein, and such ranges are understood to include each and every number and/or fraction between the stated range lower and upper values. Also, the term “about” may refer to deviations of +20% unless otherwise stated.

Embodiments relate to a shaving aid composition and a razor having the shaving aid composition. The shaving aid composition preferably includes a soap base and an effective amount of one or more polysaccharides to enhance the wear resistance of the soap base and/or the shaving aid composition. Notably, an ability of the shaving aid composition to resist wear can impact the usable life of a razor cartridge. The surprising effectiveness of certain natural and/or biodegradable polysaccharides to enhance wear resistance imparts additional sustainability benefits without sacrificing shave performance. For example, a shaving aid composition having a soap base and one or more polysaccharide wear resistance agents may provide comparable or better shave performance relative to commercially available shaving aid compositions. The shaving aid composition may also provide sustainability benefits compared to traditional technologies such as, without limitation, replacement and/or razor cartridge longevity, soap naturalness, soap biodegradability, etc.

### Razor

A shaving aid composition is erodible, having an ability to be worn away by the flow of water or as the result of abrasion. In embodiments, a shaving aid composition may be incorporated into a razor as a shaving aid body. General examples of a shaving aid body may be found in commercialized products sold under the trade name SCHICK®. Various types of shaving aid bodies are also described in United States Patent Application Publication Nos. 2005/0011073 and 2005/0015990, incorporated herein in their entirety.

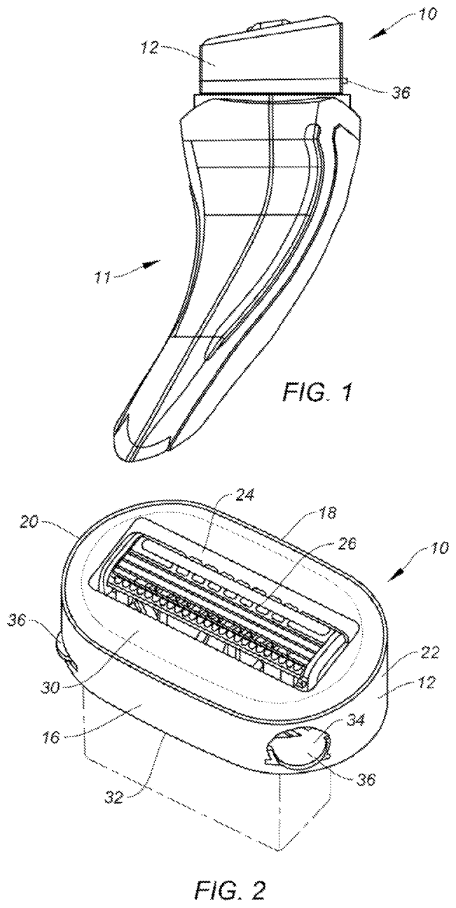

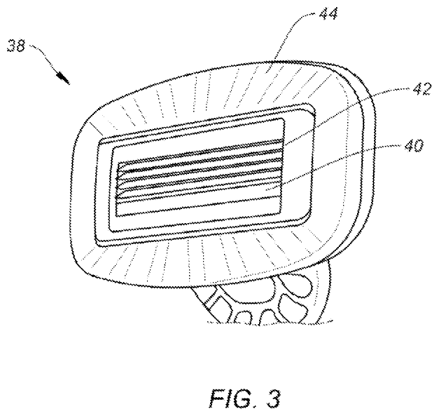

As shown in FIGS. 1-2, a replacement cartridge 10 for use in a razor 11 includes a shaving aid body 12. The shaving aid body 12 is an erodible solid body composed of a shaving aid material selected to suit the application at hand. A soap base is particularly well suited for wet shaving applications, and may include shaving aid materials (e.g., soap, lubricating agents, drag reducing agents, depilatory agents, cleaning agents, medicinal agents, sensory agents, skin stimulation agents, etc.). As shown, the shaving aid body 12 has a forward portion 16, an aft portion 18, a first lateral portion 20, a second lateral portion 22, all of which are disposed around a centrally located aperture 24, which is sized to receive a razor cartridge 26. The shaving aid body portions 16, 18, 20, 22 extend from a contact surface 30 to a base surface 32. During a shaving operation, the contact surface 30 contacts a hirsute surface as a cutting edge of the razor cartridge 26 engages the hirsute surface.

In the illustrated embodiment, the shaving aid body 12 is oval-shaped although other shapes such as, for example, circular shapes, rectangular shapes, or virtually any other geometry of the shaving aid body 12 or structural configuration of the razor 11 can be substituted without departing from the broader aspects of the present invention. For example, the shaving aid body 12 may assume an alternative shape and/or may include multiple pieces, e.g., the above-described portions of the shaving aid body 12 may be separate from one another. Similarly, while the aperture 24 is disposed in the contact surface 30, the aperture 24 may alternatively be formed by a plurality of the shaving aid portions 16, 18, 20, 22 positioned relative to one another in a manner that creates the centrally located aperture 24. Moreover, the aperture 24 may not extend between the surfaces 30, 32. In such instances, a second aperture may be included, extending between the base surface 32 and the aperture 24, having the same or different cross-sectional geometry than the aperture 24.

As shown in FIGS. 1-2, the shaving aid body 12 is coupled to a base 34. Some embodiments of the base 34 include a plurality of tabs 36 that extend out from the base 34 in a lateral direction. The tabs 36 are used to locate the replacement cartridge 10 within packaging. In one embodiment, the base 34 is integrally formed with the shaving aid body 12. The term “integrally formed” may refer to a base 34 that consists of one or more shaving aid materials or constituents thereof and is at least partially formed at the same time as the shaving aid body 12. The portion of the shaving aid material that forms the base 34 either initially has, or is processed to have, sufficient mechanical strength to be operable to enable attachment of the replacement cartridge 10 to the razor 11. The appropriate process (e.g., curing, compression, etc.) used to produce sufficient mechanical strength within the integral base 14 will depend on the shaving aid material.

As shown in FIG. 3, a shaving aid composition is incorporated into a razor 38 as a shaving aid strip 40 mounted on or adjacent to a razor cartridge 42 additionally or alternatively to a shaving aid body 44. The shaving aid strip 40 may be provided in any desired shape, location, size, portions, and so on, or combinations thereof. General examples of a shaving aid strip may be found in commercialized products sold under the trade name SCHICK®. Various types of shaving aid strips are also described in United States Patent Application Publication No. 2017/0216442, incorporated herein in its entirety.

In one embodiment, a shaving aid composition may be dispersed in a matrix of a water-insoluble material (e.g., a styrenic polymer, an olefinic polymer, a biodegradable polymer, or combinations thereof) to form a shaving aid strip that can be mounted in or on the razor cartridge 42, adjacent a cutting edge or edges. A shaving aid composition, including a soap base and one or more polysaccharides, may optionally or preferably exclude the matrix of water-insoluble material to form a shaving aid strip that can be mounted in or on the razor cartridge. In either case, a shaving aid composition including a soap base and one or more polysaccharide wear resistance agents provides good shave performance (e.g., lubrication, glide, etc.) when exposed to water or to abrasion during a shave.

### Soap Base

The soap base may be derived from a vegetable oil or tallow, saponified or neutralized, that results in the production of glycerol and salts of fatty acids (e.g., sodium carboxylate salts having up to about 22 carbon atoms) used in the soap base. Super-fatted materials containing portions (e.g., about 25 wt. % or greater) of coconut acid or other fatty acids may also be used. In one example, the soap base includes a C₁₂-C₂₂ fatty acid and/or salt(s) thereof. For example, the soap base may include lauric acid, myristic acid, palmitic acid, stearic acid, linolenic acid, or combinations thereof. The soap base may preferably include salt(s) of a C₁₂-C₂₂ fatty acid such as, without limitation, sodium, potassium, and/or lower alkanolamine salts (e.g., triethanolamine (TEA) salts, etc.) including, without limitation, sodium stearate, sodium palmitate, sodium cocoate, sodium isostearate, potassium stearate, potassium myristate, potassium palmitate, potassium cocoate, potassium isostearate, TEA-stearate, and the like, or combinations thereof. C₁₂-C₂₂ fatty acid(s) and/or salt(s) thereof may, for example, be present in an amount ≥60 wt. %, such as 65 wt. %, 70 wt. %, 75 wt. %, 80 wt. %, 85 wt. %, 90 wt. %, 95 wt. %, 99 wt. %, including every number and/or fraction therebetween, based on the total weight of the shaving aid composition. The soap base may, however, be present in an amount of about 50 wt. % to about 99.5 wt. %, preferably from about to 75 wt. % to about 99.5 wt. %, including every number and/or fraction therebetween, such as 80 wt. %, 85 wt. %, 90 wt. %, 95 wt. %, 99 wt. %, based on the total weight of the shaving aid composition.

The soap base may optionally include various additives such as, without limitation, skin care ingredients, surfactants, absorbents, colorants, fragrances, or combinations thereof. Additives may be present in an amount of about 0.001 wt. % to about 40.0 wt. %, based on the total weight of the shaving aid composition. An additive may, for example, be present in an amount of 0.0008 wt. %, 0.1 wt. %, 0.4 wt. %, 1 wt. %, 2 wt. %, 3 wt. %, 4 wt. %, 5 wt. %, 10 wt. %, 15 wt. %, 20 wt. %, 25 wt. %, 30 wt. %, 40 wt. %, 44 wt. %, including every number and/or fraction therebetween, based on the total weight of the shaving aid composition.

Skin care ingredients may include, without limitation, skin benefiting agents (e.g., emollients, lubricants, humectants, moisturizing agents, conditioners, and the like), anti-acne agents, ant-aging agents, cleansers, foaming agents, hair growth inhibitors, botanical extracts, antioxidants, antimicrobials, anti-inflammatory agents, astringents, anti-irritants, depilatory agents, medicinal agents, exfoliating agents (e.g., loofa, seaweed, oatmeal, pumice, apricot seed, and the like), or combinations thereof. A skin benefiting agent may include, without limitation, aloe barbadensis, dimethicone, allantoin, sucrose cocoate, oleyl lanolate, thiourea, cocoa butter, tocopheryl acetate, PPG-33, undeceth-3, honey, algae, or combinations thereof. Further embodiments of skin benefiting agents may include, without limitation, glycerin, glycols (e.g., propylene glycol, etc.), fatty alcohols (e.g., C₁₂-C₂₅ alcohol), adipates, benzoates, isoparaffins, esters, or combinations thereof. In one example, a skin benefiting agent may be present in an amount up to (e.g., >0 wt. % when included) 14 wt. %, preferably up to 8 wt. %, and more preferably up to about 2 wt. %, based on the total weight of the shaving aid composition. Further embodiments of skin care ingredients may include, without limitation, sodium or potassium salts (e.g., lactylates, chlorides, sulfonates, and the like), vitamins and vitamin complexes, cocoates, metal oxides, oils (e.g., cocoa butter), aloe barbadensis, extracts (e.g., plant, fruit, nut, etc.), or combinations thereof. In one example, such skin care ingredients may be present in an amount up to 10 wt. %, preferably up to 5 wt. %, and more preferably up to 3 wt. %, based on the total weight of the shaving aid composition.

Surfactants may include, without limitation, sodium isostearoyl lactylate, ammonium isostearate, DEA-myristate, alkyl glyceryl sulfonate, laureth-16, or combinations thereof. In one example, a surfactant may be present in an amount up to 9 wt. %, preferably up to 7.5 wt. %, and more preferably up to 6 wt. %, based on the total weight of the shaving aid composition. An absorbent may include, without limitation, clays or clay-based compositions, chalks, talcs, silicas, or combinations thereof. Clays that may be added include, without limitation, bentonite, kaolin, or combinations thereof. In one example, an absorbent may be present in an amount up to about 9 wt. %, preferably up to 5 wt. %, and more preferably up to about 3 wt. %, based on the total weight of the shaving aid composition.

Embodiments may also be suited for users having skin that is not sensitive to dyes or pigments used as coloring agents. Thus, a shaving aid composition may include a coloring agent such as, without limitation, titanium dioxide, manganese violet, zinc oxide, an Ultramarine (e.g., Ultramarine Blue 4), Orange 4, Green 3, or combinations thereof. In one example, a coloring agent is present in an amount up to 6 wt. %, preferably up to 4 wt. %, and more preferably up to 1 wt. %, based on the total weight of the shaving aid composition. Fragrances may also be added, which operate as odorants used to impart desirable smells to a composition and may further mask the less desirable odors of other components of a composition. In an embodiment that may be suited for users having skin that is not sensitive to fragrances, a shaving aid composition may further include a fragrance in the amount up to 2 wt. %, preferably up to 1.5 wt. %, and more preferably up to about 1 wt. %, based on the total weight of the shaving aid composition.

Preferably, the soap base is an extruded soap base or a poured soap base. Forming an extruded soap base may include using a crutching process to blend various ingredients as a secondary phase for addition to the saponification or neutralization product. After the crutching process, a drying process is employed to remove at least some of the water. The resulting dried material is then pelletized and subjected to an amalgamating process where additional ingredients may be added to produce a pelletized composition. The pelletized composition may then be refined and extruded to form an extruded shaving aid composition from extrude soap base, that can be further processed into a final desired shape.

Meanwhile, preparing a poured soap base may include reacting triglycerides in tallow or vegetable fat with a base to produce the saponification or neutralization soap product (e.g., fatty acid salts) and glycerol, and removing some water if necessary. The resulting product is purified, as the case may be, and either additional ingredients are mixed with the product or it solidifies without additional ingredients. Solidified soap without additional ingredients may, however, later be re-melted and mixed with the additional ingredients. In either case, the admixed melted soap base may be poured into a mold and allowed to cool to form a molded shaving aid composition.

Notably, the processes discussed above are non-limiting examples of traditional methods and therefore one or more aspects of the above-described methods can be readily substituted without departing from the broader aspects of the present invention. For example, preparing a poured soap base may be accomplished by dissolving corresponding fatty salts and additional ingredients in any kind of mixture of water, glycerin and/or propylene glycol. Notably, extruded soap bases and poured soap bases are readily commercially available for use in embodiments.

The molded and/or extruded shaving aid composition may include water in an amount less than 20.0 wt. %, based on a total weight of the shaving aid composition, as a substantially anhydrous shaving aid composition. In this regard, one or more ingredients of the shaving aid composition may have associated with it absorbed and/or adsorbed water, which can be carried into the otherwise substantially anhydrous shaving aid composition. For economic, sourcing, and/or formulation reasons, it may be disadvantageous to process hygroscopic materials to make them completely free of water. In addition, water that may be intentionally added can be removed to provide a substantially anhydrous shaving aid composition. Thus, for example, the shaving aid composition may include about 10.0 wt. % or less total water.

It is understood, however, that the molded and/or extruded shaving aid composition may be a substantially hydrous shaving aid composition. For example, an off-the-shelf commercially available poured soap base may have up to about 40% of water content while customized special purpose poured soap bases may have more or less water content.

Meanwhile, forming the molded and/or extruded shaving aid composition may also impact final water content. Thus, there may be less water in the molded and/or extruded shaving aid composition relative to a pre-processed state (owing to one or more raw ingredients), with water still remaining in an amount greater than 20.0 wt. %, based on a total weight of the shaving aid composition, to provide a substantially hydrous composition.

Additionally or alternatively, the shaving aid composition may be a collection of atoms or molecules held together so that, under constant dry conditions, they maintain a defined shape and size as a substantially solid shaving aid composition. It is understood that in many end uses the shaving aid composition may be exposed to water as part of its designed function such as, for example, in a shaving aid body that acts as a lubricating element when exposed to water during a shave. Thus, the shaving aid composition of a razor may be substantially anhydrous, hydrous, and/or solid before being solubilized prior to or during a shave.

### Natural Wear Resistance Agent

The natural wear resistance agent may be selected from one or more polysaccharides. Without being bound to any theory, it is believed that one or more water-soluble, natural, relatively safe, and/or biodegradable gel-building, viscosity increasing polysaccharides can help to bind water in a shaving aid composition having an extruded soap base or a melted soap base while building a flexible network that surprisingly improves wear resistance. Thus, a longer lasting soap may be provided using natural ingredients to enhance sustainability. In this regard, the shaving aid composition does not require, and may intentionally exclude, a synthetic lubricant polymer such as polyvinylpyrrolidone (PVP), polyethylene oxide (PEO), PVP/vinyl alcohol copolymer, vinyl acetate polymers and copolymers, polyacrylamides, and the like. This technical solution is particularly useful when a longer lasting soap is integrated in a replacement cartridge that serves as a lubrication and/or glide element.

In embodiments, a polysaccharide may be selected from an algae extract, a seed extract, a plant exudate, a microorganism exudate, a fruit extract, a gelling agent of animal origin, or combinations thereof. Algae extracts may include, without limitation, agar-agar, carrageenans, alginates, or combinations thereof. Seed extracts may include, without limitation, carob gum, guar gum, locust bean gum, or combinations thereof. Plant exudates may include, without limitation, gum arabicum, konjak gum, karaya gum, gum tragacanth, ghatti gum, starch or combinations thereof. Microorganism exudates may include, without limitation, xanthan gum, cellulose or its derivatives (e.g., carboxymethylcellulose, hydroxypropylcellulose, methylcellulose, hydroxypropylmethylcellulose, hydroxyethylcellulose, celluloses modified by grafting an alkyl group, etc.), pullulan, or combinations thereof. Fruit extracts may include, without limitation, pectins. Gelling agents of animal origin may include, without limitation, gelatin, caseinates, or combinations thereof. Thus, a shaving aid composition may include agar-agar, carrageenans, alginates, carob gum, guar gum, locust bean gum, gum arabicum, konjac gum, karaya gum, gum tragacanth, ghatti gum, xanthan gum, starch, cellulose or its derivatives, pectins, gelatin, and/or caseinates.

Polysaccharides may preferably be selected based on the stability or properties at specific conditions, such as at the final pH-value of the shaving aid composition. For example, xanthan gum shows gel stability in a wide range of pH-values (e.g., 1-13) while pectin rapidly degrades at room temperature at alkaline pH-values. Additionally or alternatively, polysaccharides may be selected based on the process used to manufacture the soap base. In one example, the polysaccharide used may include agar-agar when the soap base is an extruded soap base. In another example when the soap base is a poured soap base, the polysaccharide used may include agar-agar, xanthan gum, or combinations thereof. Moreover, a shaving aid composition may intentionally exclude guar gum, gummi arabicum, locust bean gum, xanthan gum, or combinations thereof. For example, a shaving aid composition may intentionally exclude one or more of the immediately foregoing polysaccharides when the soap base in the shaving aid composition is an extruded soap base.

The natural wear resistance agent may be present in an amount of about 0.25 wt. % to about 15.0 wt. %, based on the total weight of the shaving aid composition. The total concentration of polysaccharides present in a shaving aid composition may, for example, be from 0.5 wt. % to 8.0 wt. %, such as from 1.0 wt. % to 3.0 wt. %, including every number and/or fraction therebetween, based on the total weight of the shaving aid composition. Notably, mixtures of polysaccharides can be even more effective at lower concentrations. When polysaccharides in such effective amounts are combined with a soap base present in an amount of about 50 wt. % to about 99.5 wt. %, preferably from about to 75 wt. % to about 99.5 wt. %, including every number and/or fraction therebetween, based on the total weight of the shaving aid composition, a viscous gel may be created during contact with water that is substantially more viscous than traditional water/soap solutions, the polysaccharide(s) may act as a binding additive, the polysaccharide(s) may bind excessive water in solid soap, and/or the polysaccharide(s) may influence/stabilize the liquid phase of the soap.

### Example Shaving Aid Compositions

Example Table 1 shows shaving aid compositions including a soap base and a natural wear resistance agent selected from polysaccharide(s). The soap base may be an extruded soap base, which is combined with one or more polysaccharides in an extruded shaving aid composition. The extruded soap base may include, for example, sodium palmitate, sodium cocoate, sodium isostearate, isostearic acid, water, potassium palmitate, glycerin, sodium isostearoyl lactylate, potassium cocoate, potassium isostearate, sucrose cocoate, sodium chloride, polyethylene glycol (PEG)-50 shea butter, tocopheryl acetate, pentasodium pentetate, and/or tetrasodium etidronate. The soap base may also be a poured soap base, which is combined with one or more polysaccharides in a poured shaving aid composition. The poured soap base (Melt and Pour Soap) may include a commercially available soap base or special purpose soap base.

EXAMPLE TABLE 1
Shaving Aid Compositions

| | | Approx. wt. |
|---|---|---|
| Component (INCI) | Function | % |
| Example Main Ingredients | | |
| Soap Base | Soap Base | 75.0-99.5 |
| Polysaccharide(s) | Wear Resistance | 0.25-15.0 |
| | Agent | |

Various extruded shaving aid compositions and poured shaving aid compositions, including the ingredients at the corresponding approximate (+20%) concentrations shown in Table 1, were evaluate for wear resistance. The shaving aid compositions were in the form of a shaving aid body. Wear resistance was measured by maintaining a flow of water over a textured surface and between this textured surface and the shaving aid body in a water bath at a pre-selected temperature and abrading the shaving aid body to simulate a shaving process. The flow of water through the water bath may be about 0.08 gallons per minute (gpm), and the temperature of the water may be about 38° C. to about 42° C. Abrading the shaving aid body is affected by an apparatus that provides for the engagement of the shaving aid body by a textured surface and movement of the shaving aid body across the textured surface in an oscillating motion. The textured surface is preferably a stainless-steel pad having a surface finish number of 32 microinches (Ra) (about 150 grit). Each continuous movement (forward and back) of the shaving aid body over the textured surface is a cycle. One complete cycle is a movement of about six inches in each direction (forward and back). A load of 300 grams (g) is maintained on the shaving aid body using weights. Generally, the wear rate of the shaving aid body is about 0.002 to about 0.040 millimeters per cycle (mm/cycle), preferably about 0.002 to about 0.030 mm/cycle, more preferably about 0.002 to about 0.018 mm/cycle.

Example Table 2 shows the results for various extruded shaving aid compositions evaluated for wear. Examples 1A-1B are the same control sample evaluated in duplicate. The control samples consisted only of a commercially available extruded soap base and exhibited wear values of 0.0074 and 0.0088, respectively. Notably, agar-agar was surprisingly the only natural wear resistance agent tested in the extruded soap base with marked improvement in wear resistance at both the lower end and the upper end of the concentration range evaluated (Examples 2A-2D). Interestingly, there was a positive trend in wear resistance with decreasing concentration of the agar-agar in the extruded shaving aid compositions. Other polysaccharides, however, did not perform well for wear at the concentration range evaluated. Of those, only the extruded shaving aid composition having locust bean gum exhibited an improvement in wear at the upper end of the concentration range (Example 5B).

EXAMPLE TABLE 2
Wear of Extruded Shaving Aid Compositions

| | | Wear | Percent |
|---|---|---|---|
| Example | Description | mm/stroke | Change |
| 1A | Control | 0.0074 | |
| 2A | Control 1A + 1% Agar-Agar | 0.0022 | 70 |
| 2B | Control 1A + 3% Agar-Agar | 0.0017 | 77 |
| 1B | Control | 0.0088 | |
| 2C | Control 1B + 1% Agar-Agar | 0.0022 | 75 |
| 2D | Control 1B + 3% Agar-Agar | 0.0010 | 89 |
| 3A | Control 1A + 1% Guar-Gum | 0.0208 | −181 |
| 3B | Control 1A + 3% Guar-Gum | 0.0207 | −178 |
| 4A | Control 1A + 1% Gummi Arabicum | 0.0159 | −114 |
| 4B | Control 1A + 3% Gummi Arabicum | 0.0166 | −124 |
| 5A | Control 1A + 1% Locust Bean Gum | 0.0186 | −151 |
| 5B | Control 1A + 3% Locust Bean Gum | 0.0057 | 23 |
| 6A | Control 1A + 1% Xanthan Gum | 0.0179 | −142 |
| 6B | Control 1A + 3% Xanthan Gum | 0.0205 | −177 |

Example Table 3 shows the results for various poured shaving aid compositions evaluated for wear. Examples 7 and 10 are two different control samples that consisted only of poured soap base (the latter being a commercially available melt and pour soap base). The control samples exhibited wear values of 0.0187 and 0.0026, respectively. Notably, agar-agar and xanthan gum surprisingly were natural wear resistance agents that markedly improved the wear in a melt and pour soap base having a relatively low initial wear value (Examples 8-9). Interestingly, the poured shaving aid composition including agar-agar exhibited markedly improved wear considering the traditional difficulty in enhancing wear and given the relatively high initial wear value of the poured soap base (Example 11).

EXAMPLE TABLE 3
Wear of Poured Shaving Aid Compositions

| | | Wear | Percent |
|---|---|---|---|
| Example | Description | mm/stroke | Change |
| 7 | Control | 0.0187 | |
| 8 | Control 7 + 3% agar-agar | 0.0100 | 47 |
| 9 | Control 7 + 3% xanthan gum | 0.0118 | 37 |
| 10 | Control | 0.0026 | |
| 11 | Control 10 + 3% agar-agar | 0.0023 | 14 |

Accordingly, improvements in wear according to embodiments are at least comparable to, or advantageously better than, traditional technologies that attempt to address soap wear. Additionally or alternatively, the technical solutions discussed herein provide further sustainability benefits (e.g., naturalness, biodegradability, cartridge longevity, etc.) that are unmatched by traditional technologies attempting to address soap wear.

### Additional Notes and Examples

Additional examples include a shaving aid composition, a consumer product including a shaving aid composition such as a razor, a method of making a shaving aid composition, and/or a method of using a shaving aid composition.

Example 1 may include a shaving aid composition comprising a soap base and a natural wear resistant agent selected from one or more polysaccharides.

Example 2 may include the shaving aid composition of Example 1, wherein the soap base comprises a C₁₂-C₂₂ fatty acid, a salt thereof, or combinations thereof.

Example 3 may include the shaving aid composition of any of Examples 1 to 2, wherein the soap base includes lauric acid, myristic acid, palmitic acid, stearic acid, linolenic acid, a salt thereof, or combinations thereof.

Example 4 may include the shaving aid composition of any of Examples 1 to 3, wherein the soap base is present in an amount from about to 75.0 wt. % to about 99.5 wt. %, based on the total weight of the shaving aid composition.

Example 5 may include the shaving aid composition of any of Examples 1 to 4, wherein the soap base is selected from one of an extruded soap base or a poured soap base.

Example 6 may include the shaving aid composition of any of Examples 1 to 5, wherein the one or more polysaccharides are selected from an algae extract, a seed extract, a plant exudate, a microorganism exudate, a fruit extract, a gelling agent of animal origin, or combinations thereof.

Example 7 may include the shaving aid composition of any of Examples 1 to 6, wherein the one or more polysaccharides includes agar, and wherein the soap base is selected from the group consisting of an extruded soap base and a poured soap base.

Example 8 may include the shaving aid composition of any of Examples 1 to 7, wherein the one or more polysaccharides includes agar, wherein the polysaccharide intentionally excludes guar gum, gummi arabicum, locust bean gum, or xanthan gum, and wherein the soap base is selected from the group consisting of an extruded soap base and a poured soap base.

Example 9 may include the shaving aid composition of any of Examples 1 to 8, wherein the one or more polysaccharides includes xanthan gum, and wherein the soap based is selected from the group consisting of a poured soap base.

Example 10 may include the shaving aid composition of any of Examples 1 to 9, wherein the one or more polysaccharides are present in an amount from about 0.25 wt. % to about 15.0 wt. %, based on the total weight of the shaving aid composition.

Example 11 may include the shaving aid composition of any of Examples 1 to 10, wherein the one or more polysaccharides are present in an amount from about 0.5 wt. % to about 3.0 wt. %, based on the total weight of the shaving aid composition.

Example 12 may include the shaving aid composition of any of Examples 1 to 11, wherein the shaving aid composition intentionally excludes a synthetic lubricant polymer.

Example 13 may include the shaving aid composition of any of Examples 1 to 12, wherein the shaving aid composition is substantially anhydrous and solid.

Example 14 may include the shaving aid composition of any of Examples 1 to 13, wherein the shaving aid composition is in the form of a shaving aid body, a shaving aid strip, or combinations thereof.

Example 15 may include the shaving aid composition of any of Examples 1 to 14, further comprising one or more emollients, lubricants, humectants, moisturizing agents, conditioners, surfactants, emulsifiers, film formers, sunscreen actives, anti-aging actives, insect repellants, amino acids, colorants, pigments, dyes, opacifiers, pH adjusters, fragrances, fillers, vitamins and its derivatives, preservatives, preservative boosters, chelating agents, dispersing agents, stabilizers, sphingolipids, antioxidants, minerals, keratolytics, hormonal compounds, analgesics, anti-allergenic agents, H1 or H2 antihistamines, anti-inflammatory agents, anti-irritants, anti-neoplastics, immune system boosting agents, immune system suppressing agents, anti-acne agents, cleansers, anesthetics, antiseptics, skin cooling compounds, skin protectants, skin penetration enhancers, exfoliating agents, staining agents, antimicrobials, absorbents, foaming agents, hair growth inhibitors, depilatory agents, depigmenting agents, hypopigmenting agents, pharmaceutical agents, spherical powders, extracts, alpha and beta hydroxy acids, retinol and its derivatives, or combinations thereof.

Example 16 may include a razor comprising the shaving aid composition shaving aid composition of any of Examples 1 to 15.

Example 17 may include the razor of any of Examples 1 to 16, wherein the shaving aid composition is incorporated into the razor as a shaving aid body mounted on or adjacent to a razor cartridge of the razor.

Example 18 may include the razor of any of Examples 1 to 17, wherein the shaving aid composition is incorporated into the razor as a shaving aid strip mounted on or adjacent to a razor cartridge of the razor.

Example 19 may include a method to make the shaving aid composition of any of Examples 1 to 15 comprising selecting a soap base, selecting a natural wear resistant agent selected from one or more polysaccharides, and combining the soap base and the natural wear resistant agent to form the shaving aid composition.

Example 20 may include the method of any of Examples 1 to 19, wherein the soap base is selected from an extruded soap base.

Example 21 may include the method of any of Examples 1 to 19, wherein the soap base is selected from a poured soap base.

Example 22 may include a method to use a shaving aid composition of any of Examples 1 to 21 comprising contacting the shaving aid composition with one or more of a solvent to activate the shaving aid composition or a keratinous surface and/or a hirsute surface to cause abrasion to activate or aid on the activation of the shaving aid composition.

In this disclosure and the following claims, the word “or” indicates an inclusive list such that, for example, the list of X, Y, or Z means X or Y or Z or XY or XZ or YZ or XYZ. Similarly, the list of one or more of X, Y, or Z or a list of X, Y, Z, or combinations thereof means X or Y or Z or XY or XZ or YZ or XYZ. Also, the phrase “based on” is not used to represent a closed set of conditions. For example, a step that is described as “based on condition A” may be based on both condition A and condition B. In other words, the phrase “based on” shall be construed to mean “based at least in part on.” Also, the words “a” or “an” indicate “at least one.”

While the present disclosure has been particularly described, in conjunction with specific preferred embodiments, it is evident that many alternatives, modifications, and variations will be apparent to those skilled in the art in light of the foregoing description. It is therefore contemplated that the appended claims will embrace any such alternatives, modifications, and variations as falling within the true scope and spirit of the present disclosure.

What is claimed is:

1. A shaving aid composition comprising:
a soap base; and
a natural wear resistant agent selected from one or more polysaccharides.

2. The shaving aid composition of claim 1, wherein the soap base comprises a C₁₂-C₂₂ fatty acid, a salt thereof, or combinations thereof.

3. The shaving aid composition of claim 2, wherein the soap base includes lauric acid, myristic acid, palmitic acid, stearic acid, linolenic acid, a salt thereof, or combinations thereof.

4. The shaving aid composition of claim 1, wherein the soap base is present in an amount from about to 75.0 wt. % to about 99.5 wt. %, based on the total weight of the shaving aid composition.

5. The shaving aid composition of claim 1, wherein the soap base is selected from one of an extruded soap base or a poured soap base.

6. The shaving aid composition of claim 1, wherein the one or more polysaccharides are selected from an algae extract, a seed extract, a plant exudate, a microorganism exudate, a fruit extract, a gelling agent of animal origin, or combinations thereof.

7. The shaving aid composition of claim 1, wherein the one or more polysaccharides includes agar, and wherein the soap base is selected from the group consisting of an extruded soap base and a poured soap base.

8. The shaving aid composition of claim 1, wherein the one or more polysaccharides includes agar, wherein the polysaccharide intentionally excludes guar gum, gummi arabicum, locust bean gum, or xanthan gum, and wherein the soap base is selected from the group consisting of an extruded soap base and a poured soap base.

9. The shaving aid composition of claim 1, wherein the one or more polysaccharides includes xanthan gum, and wherein the soap based is selected from the group consisting of a poured soap base.

10. The shaving aid composition of claim 1, wherein the one or more polysaccharides are present in an amount from about 0.25 wt. % to about 15.0 wt. %, based on the total weight of the shaving aid composition.

11. The shaving aid composition of claim 1, wherein the one or more polysaccharides are present in an amount from about 0.5 wt. % to about 3.0 wt. %, based on the total weight of the shaving aid composition.

12. The shaving aid composition of claim 1, wherein the shaving aid composition intentionally excludes a synthetic lubricant polymer.

13. The shaving aid composition of claim 1, wherein the shaving aid composition is substantially anhydrous and solid.

14. The shaving aid composition of claim 1, wherein the shaving aid composition is in the form of a shaving aid body, a shaving aid strip, or combinations thereof.

15. The shaving aid composition of claim 1, further comprising one or more emollients, lubricants, humectants, moisturizing agents, conditioners, surfactants, emulsifiers, film formers, sunscreen actives, anti-aging actives, insect repellants, amino acids, colorants, pigments, dyes, opacifiers, pH adjusters, fragrances, fillers, vitamins and its derivatives, preservatives, preservative boosters, chelating agents, dispersing agents, stabilizers, sphingolipids, antioxidants, minerals, keratolytics, hormonal compounds, analgesics, anti-allergenic agents, H1 or H2 antihistamines, anti-inflammatory agents, anti-irritants, anti-neoplastics, immune system boosting agents, immune system suppressing agents, anti-acne agents, cleansers, anesthetics, antiseptics, skin cooling compounds, skin protectants, skin penetration enhancers, exfoliating agents, staining agents, antimicrobials, absorbents, foaming agents, hair growth inhibitors, depilatory agents, depigmenting agents, hypopigmenting agents, pharmaceutical agents, spherical powders, extracts, alpha and beta hydroxy acids, retinol and its derivatives, or combinations thereof.

16. A razor comprising the shaving aid composition of claim 1.

17. The razor of claim 16, wherein the shaving aid composition is incorporated into the razor as a shaving aid body mounted on or adjacent to a razor cartridge of the razor.

18. A method to make a shaving aid composition comprising:
selecting a soap base;
selecting a natural wear resistant agent selected from one or more polysaccharides; and
combining the soap base and the natural wear resistant agent to form the shaving aid composition.

19. The method of claim 18, wherein the soap base is selected from an extruded soap base.

20. The method of claim 18, wherein the soap base is selected from a poured soap base.
